(19) United States
(12) Patent Application Publication
(10) Pub. No.: US 2007/0093836 A1
(43) Pub. Date: Apr. 26, 2007

(54) Osteosynthesis device

(76) Inventor: Guillaume Derouet, Saint Herblain (FR)

Correspondence Address: MILES & STOCKBRIDGE PC, 1751 PINNACLE DRIVE, SUITE 500, MCLEAN, VA, 22102-3833, US

(21) Appl. No.: 11/522,522

(22) Filed: Sep. 18, 2006

(30) Foreign Application Priority Data
Sep. 19, 2005 (FR) 05 9523

(51) Int. Cl. A61F 2/30

(52) U.S. Cl. 606/69

(57) ABSTRACT

The subject of the invention is an osteosynthesis device comprising a plate (2) provided with screw holes and screws (3), characterized in that at least one of the screw holes (4) is shaped so as to form a tool (5) that will form a helical groove on the proximal part (6) of a screw (3).

### FIELD OF THE INVENTION

The invention relates to an osteosynthesis device.

### BACKGROUND OF THE INVENTION

In a more specific but nonlimiting way, it relates to osteosynthesis devices used for microsurgery.

When there is a fracture, it is often necessary to hold the bones in place by means of a plate that is fastened along the fractured bone for the time required for the formation of the bone and the consolidation of the fracture.

The plate is, of course, adapted to the morphology of the bone that it must set.

Screws screwed into screw holes in the plate fasten the osteosynthesis plate to the bone fragments.

This plate must prohibit the movement of one bone fragment relative to another, since that would prevent proper consolidation of the fracture.

For this reason, it is advantageous to anchor the screw to the plate.

In one way of anchoring the screw to the plate, the latter comprises, in addition to the conventional thread of the screw that must penetrate into the bone, a supplementary screw thread for engaging with the one that lines the screw hole.

The screw in this case must be inserted in a predefined direction during the construction of the osteosynthesis plate. This direction is defined by the orientation of the screw thread in the plate; otherwise, it is necessary to use more complicated, bulkier systems.

Depending on the location of the fracture, the features of this plate can be quite different.

In essence, a plate placed on a phalange is not subjected to the same stress as a plate placed on a radius.

For these relatively small bones, it is necessary for the plate to be relatively thin. This is made possible by the fact that they are subjected to much lower stresses.

It is also important to be able to orient the screw in different directions.

This is a substantial problem for plates that are not very thick.

The currently known structures for thicker plates are inappropriate.

However, there is a known osteosynthesis device (WO-A-2004/032751) that comprises a plate and screws.

This plate has, at the level of the screw holes, a ring-shaped insert made of soft material that can be tapped by a screw comprising at its end located nearest the head of said screw a tapping thread capable of creating a complementary helical groove in this soft material.

The screw can therefore be screwed into the screw hole in an angular orientation that is not predetermined, and this screw will form a helical groove in the plastic material.

This makes it possible to anchor the screw into the plastic ring.

This solution seems advantageous, but if a screw is inadvertently inserted in the wrong direction, the ring, thus tapped, can no longer receive a screw in a second direction.

The plate must therefore be replaced, which is unsatisfactory.

Furthermore, in mounting this insert to the plate, an excess thickness is necessarily created for securing the insert.

### SUMMARY OF THE INVENTION

The object of the invention is to provide a solution.

To this end, the subject of the invention is an osteosynthesis device comprising a plate with screw holes, this device being characterized in that the periphery of at least one of the screw holes is shaped so as to form a tool that will form a helical groove on the proximal part of a screw.

### BRIEF DESCRIPTION OF THE DRAWING

The invention will be more clearly understood with the aid of the description below, given as a nonlimiting example in reference to the drawing, which represents:

FIG. 1: An osteosynthesis plate

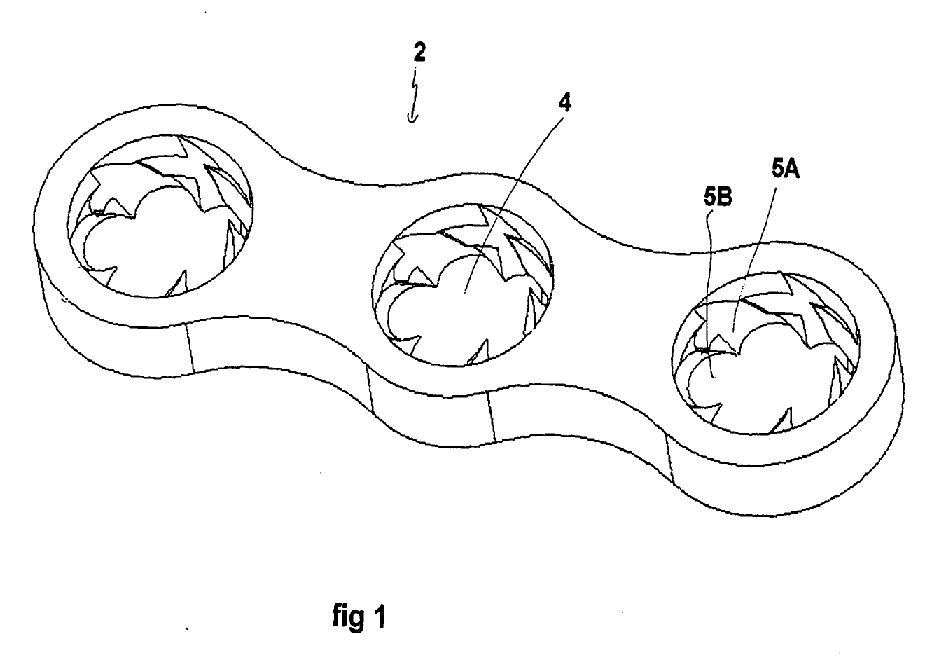

FIG. 2: An osteosynthesis plate and its screw

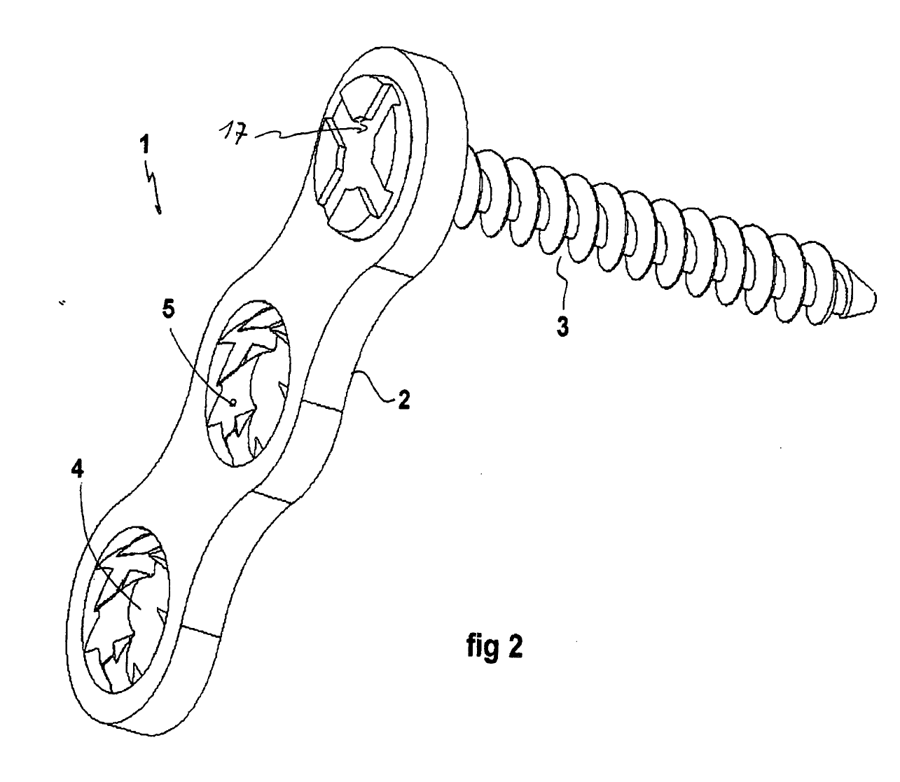

FIG. 3: A screw

### DESCRIPTION OF THE PREFERRED EMBODIMENTS

Referring to the drawing, we see an osteosynthesis device 1, for example for maintaining two bone fragments. This device comprises a plate 2 shaped in accordance with its intended use and screws 3.

The main object is to treat the small bones of the hand or the foot. This plate is therefore of small thickness.

This plate includes screw holes 4.

According to the invention, at least one of the screw holes 4 is shaped so as to form a tool 5 that will form a helical grove in the proximal part 6 of a screw 3.

The proximal part is understood to mean the part located at the level of the head of the screw.

The helical groove will be formed on all or part of this head.

This tool 5, also called a screwing die, will either remove material in order to form a helical groove on the proximal part 3 of the screw, or deform this material.

At least the zone of the head of the screw to be screwed in is less hard than the material of the tool 5 formed or mounted in the plate 2.

Prior to use, the screw is in the form of a cylindrical body whose distal part 7 has a screw thread that will be screwed into the bone. The proximal part 6, prior to use, is smooth and hence lacks a helical groove.

In one embodiment, the part of the screw that must be cut into by the tool 5 is overmolded onto the body of the screw. For example, the proximal part of the screw is made of polyetheretherketone (PEEK).

In this zone 6, called the overmolded zone, the body of the screw is of smaller cross section.

The overmold will therefore be attached axially. Forms make it possible to apply the overmold in rotation.

With this solution, even if the screw is inserted along an unsuitable axis, it need only be replaced by an unused screw.

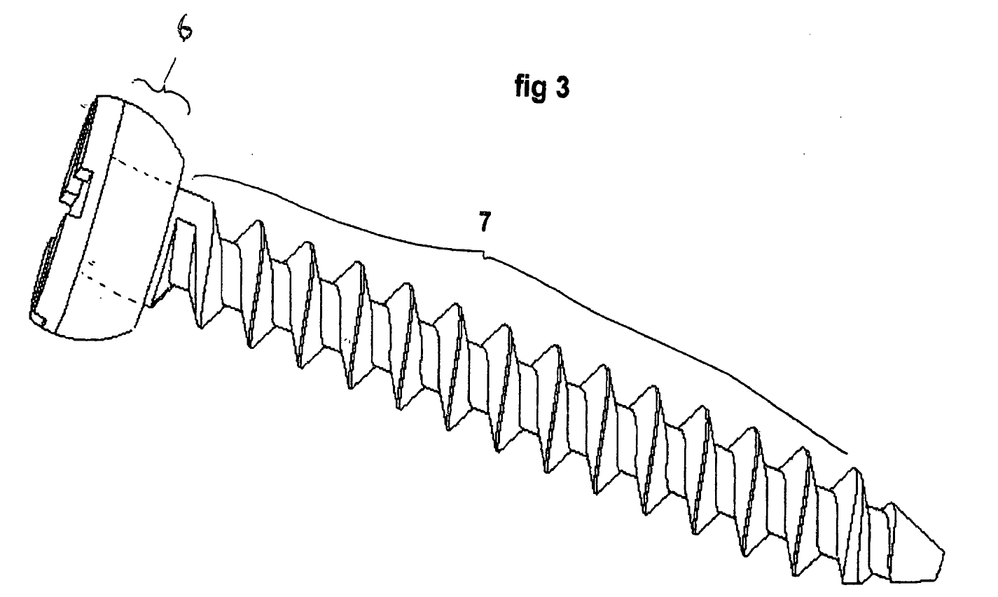

FIG. 3 shows the unused screw, and it has no helical groove.

As we can see, the proximal part 6 of the screw is in the approximate form of a hemisphere located underneath the slot 17 used to drive the screw. The tool 5 has teeth 5A distributed along the circumference of the screw hole. These teeth protrude from an approximately hemispherical internal surface. They are cut and inset so as to form a helix of a given pitch and a given profile.

Between two teeth 5A, a cutout 5B is provided for the evacuation of the chip formed during the creation of the helical groove.

Thus it is possible to orient the screw in directions other than the normal to the plane of the screw hole.

The pitch formed by the tool 5 can be different from that of the screw that screws into the bone so as to be able to anchor the osteosynthesis plate to the bone.

The plate and/or the screw are made of a biocompatible material such as stainless steel or titanium.

1. Osteosynthesis device comprising a plate (2) having a plurality of screw holes, at least one of the screw holes (4) having a shaped interior forming a tool (5), said tool being adapted to form a helical groove on a proximal part (6) of a head of a screw (3) when screwed into the said at least one hole.

2. Osteosynthesis device according to claim 1, characterized in that the tool (5) is disposed within said at least one of the screw holes in the osteosynthesis plate (2).

3. Osteosynthesis device according to claim 1, characterized in that the tool is of a material compositions that is harder than at least the proximal part of the head of the screw to be screwed into the screw hole.

4. Osteosynthesis device according to claim 3, characterized in that the proximal part (6) of the head of the screw is overmolded onto a body portion of the screw.

5. Osteosynthesis device according to claim 4, characterized in that the proximal part (6) of the screw is made of polyetheretherketone (PEEK).

6. Osteosynthesis device according to claim 1, characterized in that:
the proximal part (6) of the screw is in the approximate shape of a hemisphere located underneath a slot (7) in the head of the screw used to drive the screw into the tool, and
the tool (5) includes teeth (5A) distributed along the internal circumference of the screw hole, said teeth protruding from an approximately hemispherical internal surface.

7. Osteosynthesis device according to claim 6, characterized in that a cutout is disposed between the teeth (5A).

8. Osteosynthesis device according to claim 1, including a tool disposed within at least one of the screw rolls and a screw adapted to be screwed into said at least one screw hole, the screw having a head and a slot in the head,
the proximal part (6) of the screw is in the approximate shape of a hemisphere located underneath the slot (7) in the head of the screw, and
the tool (5) further includes teeth (5A) distributed along the internal circumference of the screw hole, said teeth protruding from an approximately hemispherical internal surface.

9. Osteosynthesis device according to claim 8, characterized in that a cutout is provided between the teeth (5A) for removal of chips.

10. Osteosynthesis device according to claim 3, including a tool disposed within at least one of the screw holes and a screw adapted to be screwed into said at least one screw hole, the screw having a head and a slot in the head,
the proximal part (6) of the screw is in the approximate shape of a hemisphere located underneath a slot (7) in the head of the screw used to drive the screw, and
the tool (5) further includes teeth (5A) distributed along the internal circumference of the screw hole, said teeth protruding from an approximately hemispherical internal surface.

11. Osteosynthesis device according to claim 10, characterized in that a cutout is provided between the teeth (5A) for removal of chips.

12. Osteosynthesis device according to claim 4, including a tool disposed within at least one of the screw holes and a screw adapted to be screwed into said at least one screw hole, the screw having a head and a slot in the head,
the proximal part (6) of the screw is in the approximate shape of a hemisphere located underneath a slot (7) in the head of the screw used to drive the screw, and
the tool (5) further includes teeth (5A) distributed along the internal circumference of the screw hole, said teeth protruding from an approximately hemispherical internal surface.

13. Osteosynthesis device according to claim 12, characterized in that a cutout is provided between the teeth (5A) for removal of chips.

14. Osteosynthesis device according to claim 5, including a tool disposed within at least one of the screw holes and a screw adapted to be screwed into said at least one screw hole, the screw having a head and a slot in the head,
the proximal part (6) of the screw is in the approximate shape of a hemisphere located underneath a slot (7) in the head of the screw used to drive the screw, and
the tool (5) further includes teeth (5A) distributed along the internal circumference of the screw hole, said teeth protruding from an approximately hemispherical internal surface.

15. Osteosynthesis device according to claim 13, characterized in that a cutout is provided between the teeth (5A) for removal of chips.
